(19) United States
(12) Patent Application Publication
(10) Pub. No.: US 2012/0142993 A1
(43) Pub. Date: Jun. 7, 2012

(54) Method for large scale production of cesium-131 with low cesium-132 content

(75) Inventors: Sergey Zlokazov, Zarechnyi (RU); David J. Swanberg, Kennewick, WA (US); Oleg Egorov, West Richland, WA (US); Garrett N. Brown, Richland, WA (US); Dale E. Boyce, Richland, WA (US)

(73) Assignee: IsoRay Medical, Inc., Richland, WA (US)

(21) Appl. No.: 11/712,594

(22) Filed: Feb. 27, 2007

Related U.S. Application Data
(60) Provisional application No. 60/777,487, filed on Feb. 28, 2006.

(51) Int. Cl. A61N 5/00; C22C 24/00; G21G 1/06

(52) U.S. Cl. 600/8; 376/189; 420/400

(57) ABSTRACT

The present invention provides a method for large scale production of cesium-131 (Cs-131) with low cesium-132 (Cs-132) content, where the Cs-131 is produced via barium-131 (Ba-131) decay. Uses of the Cs-131 produced by the method include cancer research and treatment, such as for use in brachytherapy. Cesium-131 is particularly useful in the treatment of faster growing tumors.

### CROSS REFERENCE TO RELATED APPLICATION

This application claims the benefit under 35 U.S.C. §119(e) of U.S. Provisional Patent Application No. 60/777,487 filed Feb. 28, 2006, where this provisional application is incorporated herein by reference in its entirety.

### BACKGROUND OF THE INVENTION

1. Field of the Invention

The present invention relates generally to a method for large scale production of cesium-131 (Cs-131) with low cesium-132 (Cs-132) content, where the Cs-131 is produced via barium-131 (Ba-131) decay. Uses of the Cs-131 purified by the method include cancer research and treatment, such as for use in brachytherapy implant seeds independent of method of fabrication.

2. Description of the Related Art

Radiation therapy (radiotherapy) refers to the treatment of diseases, including primarily the treatment of tumors such as cancer, with radiation. Radiotherapy is used to destroy malignant or unwanted tissue without causing excessive damage to the nearby healthy tissues.

Ionizing radiation can be used to selectively destroy cancerous cells contained within healthy tissue. Malignant cells are normally more sensitive to radiation than healthy cells. Therefore, by applying radiation of the correct amount over the ideal time period, it is possible to destroy essentially all of the undesired cancer cells while saving or minimizing damage to the healthy tissue. For many decades, localized cancer has often been cured by the application of a carefully determined quantity of ionizing radiation during an appropriate period of time. Various methods have been developed for irradiating cancerous tissue while minimizing damage to the nearby healthy tissue. Such methods include the use of high-energy radiation beams from linear accelerators and other devices designed for use in external beam radiotherapy.

Another method of radiotherapy includes brachytherapy. Here, radioactive substances in the form of seeds, needles, wires or catheters are implanted permanently or temporarily directed into/near the cancerous tumor. Historically, radioactive materials used have included radon, radium and iridium-192. More recently, the radioactive isotopes Cs-131, iodine-125 (I-125), and palladium-103 (Pd-103) have been used. Examples are described in U.S. Pat. Nos. 3,351,049; 4,323,055; and 4,784,116.

During the last 30 years, numerous articles have been published on the use of I-125 and Pd-103 in treating prostate cancer. Despite the demonstrated success in certain regards of I-125 and Pd-103, there are certain disadvantages and limitations in their use. While the total dose can be controlled by the quantity and spacing of the seeds, the dose rate is set by the half-life of the radioisotope (60 days for I-125 and 17 days for Pd-103). For use in faster growing tumors, the radiation should be delivered to the cancerous cells at a faster rate, while simultaneously preserving all of the advantages of using a soft x-ray emitting radioisotope. Such cancers are those often found in the brain, lung, pancreas, prostate and other tissues.

Cesium-131 (Cs-131) is a radionuclide product that is ideally suited for use in brachytherapy (cancer treatment using interstitial implants, i.e., “radioactive seeds”). The short half-life of Cs-131 makes the seeds effective against faster growing tumors such as those found in the brain, lung, and other sites. While prostate cancer is generally considered slower growing, certain prostate cancers are more aggressive and more appropriately treated using an isotope with a shorter half-life such as Cs-131. The shorter half-life of Cs-131 is equally effective against the slower growing tumors and thus is applicable for treatment where the aggressiveness of the tumor is not well known in advance (C.I. Armpilia et al., Int. J. Radiat. Oncol. Biol. Phys. 55:378-385 (2003)).

Cesium-131 is produced by radioactive decay from neutron irradiated naturally occurring Ba-130 (natural Ba comprises about 0.1% Ba-130) or from enriched barium containing additional Ba-130, which captures a neutron, becoming barium-131 (Ba-131). The source of the neutrons can be a nuclear reactor or other neutron generating devices (e.g., neutron generators). Barium-131 then decays with an 11.7-day half-life to cesium-131, which subsequently decays with a 9.7-day half-life to stable xenon-130. Thus, with the decay of Ba-131 comes the buildup of Cs-131. To separate the Cs-131, the barium target is “milked” multiple times over selected intervals such as 7 to 14 days, as Ba-131 decays to Cs-131. With each “milking”, the Curies of Cs-131 present and gram ratio of Cs to total Ba decreases (less Cs-131 per gram of Ba) until it is not economically of value to continue to “milk the cow” (e.g., after approximately 40 days). The barium “target” can then be returned to the reactor for further irradiation (if sufficient Ba-130 is present) or discarded.

In order for the Cs-131 product to be useful, the overall content of Cs-132 in the Cs-131 product must be kept as low as possible. Cesium-132 is an unwanted impurity in the Cs-131 product. As described above, Cs-131 is typically produced via irradiation of Ba targets with neutrons to yield Ba-131 which then decays to Cs-131. During irradiation of Ba targets with neutrons, the Cs-131 isotope produced (via Ba-131 decay) during the irradiation period can further interact with neutrons to yield Cs-132. Because of the decay characteristics of Cs-132, its presence in the final Cs-131 product is undesirable.

Once Cs-132 is present in the final product, Cs-132 cannot be separated from Cs-131 by common chemical separation methods. In order to minimize the level of Cs-132 impurity, the typical approach in the art is the selection of neutron irradiation conditions (i.e., neutron flux and irradiation time) that minimize production yields of Cs-132 in the target material. However, the problem is that irradiation conditions that minimize production of Cs-132 can lead to reduction of the overall amount of Ba-131 produced in the target. Consequently, the overall amount of the Cs-131 that can be obtained from the irradiated target over several cycles of Cs-131 ingrowth and separation can be reduced.

Due to the need for, and the deficiencies in the art for producing, Cs-131 with low Cs-132 content but without compromising the overall amount of Cs-131 obtainable from the irradiated target, there is a need for improved methods for large scale production of Cs-131 with low Cs-132 content. The present invention fulfills this need and provides further related advantages.

### BRIEF SUMMARY OF THE INVENTION

Briefly stated, the present invention discloses a method for large scale production of Cs-131 with low Cs-132 content. The method is useful, for example, for producing Cs-131 with low Cs-132 on a large commercial scale in a manner optimized for utilization of Cs-131 in cancer treatment.

In one embodiment, the method for the large scale production of Cs-131 with low Cs-132 content comprises the steps of: (a) irradiating a barium (Ba) target with neutrons, whereby irradiated target containing Ba-131 and cesium is produced; (b) separating the cesium from the irradiated barium target between about six hours to five days following the termination of irradiation, thereby substantially removing Cs-132 from the irradiated barium target; (c) allowing less than fifteen days for the ingrowth of Cs-131 in the irradiated barium target of step (b), whereby the Cs-131 is produced via decay of Ba-131 in the irradiated target; and (d) separating the Cs-131 and the irradiated barium target, thereby effecting large scale production of Cs-131 with low Cs-132 content.

In an embodiment, step (b) is performed about six to twelve hours following termination of irradiation.

In an embodiment, step (c) is performed for about one to twelve days.

In an embodiment, step (c) is performed for about seven days.

In an embodiment, steps (c) and (d) are reperformed one or more additional times.

In an embodiment, steps (c) and (d) are reperformed fourteen or less days after step (d).

In an embodiment, steps (c) and (d) are reperformed seven or less days after step (d).

In an embodiment, steps (c) and (d) are reperformed twenty-four hours after step (d).

In an embodiment, step (b) is repeated prior to step (c) to remove residual Cs-132.

In an embodiment, the repetition of step (b) is immediately after the conclusion of step (b).

In an embodiment, steps (a) through (d) are performed on a second barium target, and the Cs-131 of step (d) from the initial barium target and from the second barium target are combined.

In an embodiment, steps (a) through (d) are performed on each of three or more barium targets, and the Cs-131 of step (d) from the initial barium target and from each of the three or more step (d) are combined.

The present invention provides purified Cs-131 with low Cs-132 content comprising Cs-131 prepared by a method of the present invention.

The present invention provides a radioactive brachytherapy implant substance comprising a brachytherapy implant substance containing Cs-131 with low Cs-132 content prepared by a method of the present invention.

These and other aspects of the present invention will become apparent upon reference to the following detailed description.

### DETAILED DESCRIPTION OF THE INVENTION

The present invention provides a method of producing Cs-131 on a large scale with low Cs-132 content that optimizes the amount of Cs-131 that can be utilized in practice. The method provides for large scale continuing production of multi Ci quantities of Cs-131, rather than to create a single batch with the highest activity possible. The method is efficient and economical, as the irradiation conditions are not altered to minimize the production of Cs-132 and thus the overall amount of Cs-131 is not compromised by altered irradiation conditions. In other words, the target irradiation conditions can be therefore optimized for maximum production of Ba-131, without concerns over the levels of Cs-132 buildup in the target. Thus the target can be irradiated over longer periods of time which makes this production procedure compatible with reactors operating on longer cycles.

The neutron irradiation of a barium target is well known to one in the art (e.g., Harper, P. V. et al., Proceedings of the International Conference on the Peaceful Uses of Atomic Energy, 2nd, Geneva, Switzerland, 1958, pp. 417-422). A typical target size ranges from several grams to several kilograms of natural Ba. However, for the large scale production of Cs-131 by the method of the present invention, typically the target size is a minimum of 100 grams of natural Ba or, if enriched, at least the equivalent of 0.1 grams of Ba-130 (0.1% to 100% enrichment; natural Ba comprises about 0.1% Ba-130). Such a target size yields at least 10 Ci per week or 40 Ci per month of Cs-131. For large scale production, yields of several hundred Ci per week are preferred. Typically, irradiated Ba targets comprise various Ba salts. Most often Ba carbonate is used.

In one embodiment, the cesium (e.g., Cs-131 and Cs-132) contained in the irradiated barium target is separated from the Ba target material between about six hours to five days following the termination of irradiation. Thus, the separation generally occurs shortly after the end of irradiation. The chemical purity of the barium target is optimized so that a long cooling down period after irradiation is not necessary. Typically the separation is performed from about six hours to twelve hours after the end of irradiation. The cesium is produced when a barium (Ba) target (i.e., containing Ba-130) is irradiated with neutrons, and the Ba-130 captures a neutron becoming Ba-131 which then decays to Cs-131. However, if the irradiation with neutrons has not ended prior to the creation of Cs-131 (via Ba-131 decay), then the Cs-131 can further interact with neutrons to yield Cs-132. By separating the cesium from the irradiated barium target, shortly following the cessation of neutron irradiation, the Cs-131 that is then formed by the decay of Ba-131 after irradiation has terminated will exist in the absence of neutrons which could convert some of the Cs-131 to Cs-132.

In another embodiment, the separation of cesium from the irradiated barium target is repeated to remove residual Cs-132. This separation may occur any time up to about five days after the initial separation. Typically, the repeat of separation of cesium from the irradiated barium target is performed immediately after the initial separation.

Following removal of the cesium (which is normally discarded due to Cs-132 content), the irradiated barium target is set aside for less than fifteen days to allow for the ingrowth of Cs-131 in the irradiated barium target. As described above, the decay of Ba-131 (produced by neutron irradiation of Ba-130) yields Cs-131. The time to allow ingrowth of Cs-131 in the irradiated barium target is typically for the present invention about one to twelve days, with one week (seven days) being a useful and convenient time interval.

Once the desired time has passed for the ingrowth of Cs-131, it is separated from the irradiated barium target. As used herein, “separating the Cs-131” may mean removing the Cs-131 from the irradiated barium target, or removing the irradiated barium target from the Cs-131, or removal of both simultaneously. In addition, as used herein, the irradiated barium target may have been partially purified prior to separating the Cs-131. Procedures for separating Cs-131 from irradiated barium targets are well known in the art (e.g., U.S. Pat. No. 6,066,302). For example, chemical separation steps can be utilized to isolate Cs-131 from the target material and radioactive impurities that may have been produced in the target material during irradiation.

Following separation of the Cs-131 and the irradiated barium target, the ingrowth and separation steps may be repeated. More specifically, following the separation of the irradiated barium target and the Cs-131, the irradiated barium target is set aside to permit accumulation of additional Cs-131 from the further decay of Ba-131. The additional Cs-131 and irradiated barium target are separated. The repeated ingrowth and separation are generally performed fourteen or less days after the initial separation. Typically the repeated steps are performed seven or less days after the initial separation. In an embodiment, the repeated steps are performed twenty-four hours after the initial separation. The ingrowth and separation steps may be repeated more than once (one or more additional times).

The entire procedure (from irradiation of a barium target to the final separated Cs-131) may be performed on a second barium target. The second barium target may be more recently irradiated. The Cs-131 from the first barium target and the second barium target may be combined. Similarly, the entire procedure may be performed on each of three or more barium targets, and the Cs-131 from the initial barium target and from each of the three or more Cs-131 separation steps may be combined. The pooling from two or more irradiated barium targets of Cs-131 with low Cs-132 content aids large scale production.

As described above, Cs-131 is useful for example for radiotherapy (such as to treat malignancies). Where it is desired to implant a radioactive substance (e.g., Cs-131) into/near a tumor for therapy (brachytherapy), Cs-131 may be used as part of the fabrication of brachytherapy implant substance (e.g., seed). A brachytherapy implant substance containing Cs-131 may be incorporated into a brachytherapy device. The use of Cs-131 in brachytherapy implant substances or devices is not dependent on the method of fabrication of the substances. A method of the present invention provides purified Cs-131 for these and other uses.

The following Examples are offered by way of illustration and not by way of limitation.

### EXAMPLES

### Example 1

A single irradiated Ba target is processed 6 hours after the end of irradiation and the separated Cs is discarded. The target is then milked repeatedly using 7-day intervals.

1. Irradiate 1 kg natural BaCO₃ target (or 10 g of enriched BaCO₃; 10% Ba-130 enrichment) in a nuclear reactor at neutron flux of ˜4.1×10¹⁴ neutrons cm⁻² s⁻¹ for a period of two weeks. The Ba-131 activity in the target at the end of irradiation is ˜100 Ci.

2. Transfer target from the irradiation facility to the remote chemical processing facility and perform Cs/Ba separation. Time after the end of irradiation is 6 hours. The ratio of Cs-132 to Cs-131 in separated Cs-131 is 1.2%. Chemical removal efficiency of Cs is 90% or greater. Discard separated Cs and dispose of as radioactive waste.

3. Repeat Cs/Ba separation (step 2) to ensure more complete removal of Cs-132.

4. Set processed target aside for Cs-131 accumulation period of 7 days.

5. Perform Cs/Ba separation. Time after irradiation is 7 days. Chemical recovery of Cs is 90%. The activity of separated Cs-131 is 28 Ci. The ratio of Cs-132 to Cs-131 is 0.008%.

6. Repeat steps 4 and 5. Time after irradiation is 14 days. Chemical recovery of Cs is 90%. The activity of separated Cs-131 is 19 Ci. The ratio of Cs-132 to Cs-131 is 0.001%.

7. Repeat steps 4 and 5. Time after irradiation is 21 days. Chemical recovery of Cs is 90%. The activity of separated Cs-131 is 12 Ci. The ratio of Cs-132 to Cs-131 is 0.0001%.

8. Repeat steps 4 and 5. Time after irradiation is 28 days. Chemical recovery of Cs is 90%. The activity of separated Cs-131 is 8 Ci. The ratio of Cs-132 to Cs-131 is <0.0001% (Cs-132 is below detection limits of the analysis method).

9. Repeat steps 4 and 5. Time after irradiation is 35 days. Chemical recovery of Cs is 90%. The activity of separated Cs-131 is 5 Ci. The ratio of Cs-132 to Cs-131 is <0.0001% (Cs-132 is below detection limits of the analysis method).

10. Discard decayed Ba target or recycle for re-irradiation. Total amount of produced Cs-131 over period of 35 days of milking is 72 Ci.

### Example 2

A single irradiated Ba target is processed 6 hours after end of irradiation and separated Cs is discarded. The target is milked using 14-day intervals for the period of 28 days. The Cs-131 activity is in equilibrium with Ba-131 activity. The total amount of activity produced is given below.

1. Irradiate 1 kg natural BaCO₃ target (or 10 g of enriched BaCO₃; 10% Ba-130 enrichment) in a nuclear reactor at neutron flux of ˜4.1×10¹⁴ neutrons cm⁻² s⁻¹ for a period of two weeks. The Ba-131 activity in the target at the end of irradiation is ˜100 Ci.

2. Transfer target from the irradiation facility to the remote chemical processing facility and perform Cs/Ba separation. Time after the end of irradiation is 6 hours. The ratio of Cs-132 to Cs-131 in separated Cs-131 is 0.12%. Chemical recovery of Cs is 90%. Discard separated Cs and dispose of as radioactive waste.

3. Repeat Cs/Ba separation (step 2) to ensure complete removal of Cs-132.

4. Set processed target aside for Cs-131 accumulation period of 14 days.

5. Perform Cs/Ba separation. Time after irradiation is 14 days. Chemical recovery of Cs is 90%. The activity of separated Cs-131 is 36 Ci. The ratio of Cs-132 to Cs-131 is 0.008%.

6. Repeat steps 4 and 5. Time after irradiation is 28 days. Chemical recovery of Cs is 90%. The activity of separated Cs-131 is 15 Ci. The ratio of Cs-132 to Cs-131 is 0.001%.

7. Discard decayed Ba target or recycle for re-irradiation. Total amount of produced Cs-131 over period of 28 days of milking is 51 Ci.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A method for the large scale production of Cs-131 with low Cs-132 content, comprising the steps of:
(a) irradiating a barium (Ba) target with neutrons, whereby irradiated target containing Ba-131 and cesium is produced;
(b) separating the cesium from the irradiated barium target between about six hours to five days following the termination of irradiation, thereby substantially removing Cs-132 from the irradiated barium target;
(c) allowing less than fifteen days for the ingrowth of Cs-131 in the irradiated barium target of step (b), whereby the Cs-131 is produced via decay of Ba-131 in the irradiated target; and
(d) separating the Cs-131 and the irradiated barium target, thereby effecting large scale production of Cs-131 with low Cs-132 content.

2. The method of claim 1 whereby step (b) is performed about six to twelve hours following termination of irradiation.

3. The method of claim 1 whereby step (c) is performed for about one to twelve days.

4. The method of claim 3 whereby step (c) is performed for about seven days.

5. The method of claim 1 whereby steps (c) and (d) are reperformed one or more additional times.

6. The method of claim 5 whereby steps (c) and (d) are reperformed fourteen or less days after step (d).

7. The method of claim 5 whereby steps (c) and (d) are reperformed seven or less days after step (d).

8. The method of claim 5 whereby steps (c) and (d) are reperformed twenty four hours after step (d).

9. The method of claim 1 whereby step (b) is repeated prior to step (c) to remove residual Cs-132.

10. The method of claim 9 whereby the repetition of step (b) is immediately after the conclusion of step (b).

11. The method of claim 1 whereby steps (a) through (d) are performed on a second barium target, and the Cs-131 of step (d) from the initial barium target and from the second barium target are combined.

12. The method of claim 1 whereby steps (a) through (d) are performed on each of three or more barium targets, and the Cs-131 of step (d) from the initial barium target and from each of the three or more step (d) are combined.

13. Purified Cs-131 with low Cs-132 content comprising Cs-131 prepared by the method of claim 1, 11 or 12.

14. A radioactive brachytherapy implant substance comprising a brachytherapy implant substance containing the Cs-131 with low Cs-132 content of claim 13.
